(19) United States
(12) Patent Application Publication
(10) Pub. No.: US 2015/0110768 A1
(43) Pub. Date: Apr. 23, 2015

(54) COMPOSITIONS AND METHODS COMPRISING SIRTUIN STIMULATORS AND NON-SIRTUIN PROTEIN DEACETYLASE INHIBITORS

(71) Applicant: Biocogent, LLC, Stony Brook, NY (US)

(72) Inventor: Joseph D. Ceccoli, Farmingville, NY (US)

(21) Appl. No.: 14/518,860

(22) Filed: Oct. 20, 2014

Related U.S. Application Data
(60) Provisional application No. 61/951,256, filed on Mar. 11, 2014.
(60) Provisional application No. 61/892,867, filed on Oct. 18, 2013.

(51) Int. Cl. A61K 38/50; A61K 31/05

(52) U.S. Cl. CPC A61K 38/50; A61K 31/05; C12Y 305/01098
USPC 424/94.6

(57) ABSTRACT

The present technology relates to cosmetic and pharmaceutical compositions useful for minimizing the appearance of aging via a safe method of stimulating sirtuin activity. In particular, the present technology relates to compositions and methods that utilize a combination of a sirtuin activator with a sirtuin-offsetting agent in order to maintain a healthy population of skin cells while preventing survival and growth of abnormal cells that are irreparably damaged.

### BACKGROUND

The present technology relates to cosmetic and pharmaceutical compositions useful for controlling the rate of cell destruction and minimizing the appearance of aging. In particular, the present technology relates to compositions and methods comprising a combination of a sirtuin activator with a sirtuin-offsetting agent.

Sirtuins are a class of protein deacetylase enzymes that require nicotinamide adenine dinucleotide (NAD) as a cofactor in deacetylating lysine residues in target proteins. Acetylation and deacetylation of certain amino acids is a post-translational modification that controls the activities of some target proteins. The targets that were initially discovered were the histone proteins that package DNA in cell nuclei; thus, these enzymes are commonly referred to as histone deacetylases (HDACs), despite the fact that many non-histone target proteins have been discovered subsequent to the initial discoveries.

Sirtuins have been found to influence various biological phenomena, including cellular stress responses such as DNA repair, replicative senescence and apoptosis (suicide or “programmed cell death” response that cells typically undergo following serious or irreparable damage). Specifically, sirtuins have been found to delay apoptosis of damaged cells, thereby slowing down or eliminating their destruction.

There is concern that the mechanism through which sirtuins may slow down aging may possess an inherently dangerous side effect—specifically, the apoptotic response protects organisms by eliminating damaged cells that could otherwise become genetically unstable and lose normal growth controls and proper differentiated function. By having the effect of delaying apoptosis of damaged cells, sirtuins might allow them to escape normal checks, resulting in overgrowth of tissues with abnormal progeny cells, which would compromise proper tissue function and possibly even imperil organism survival.

Thus, a need exists for “safe” sirtuin compounds and methods and functional activity—specifically, compositions that harness the anti-aging benefits of sirtuins without the undesirable side effects; as well as methods of optimizing such compositions and of using them in anti-aging applications for patients.

### SUMMARY OF THE DISCLOSED TECHNOLOGY

In certain embodiments, the present technology is directed to compositions comprising a sirtuin activator (also referred to herein as a sirtuin stimulator) as well as a sirtuin-offsetting agent.

In certain embodiments, the present technology is directed to methods of formulating a composition, or of optimizing the efficacy of a composition for a patient, or of optimizing cell maintenance in a patient, the methods comprising selecting a sirtuin stimulator having a known quantitative prolonging effect on a cell, and selecting a sirtuin-offsetting agent having a known quantitative opposite effect on the cell, and optimizing the balance between the two effects based on a known desired ultimate effect on the cell.

In certain embodiments, the present technology is directed to a method of prolonging the life of a cell and simultaneously avoiding proliferation of cell damage, the method comprising the steps of: stimulating Sirt1 activity in the cell; and inhibiting a non-sirtuin HDAC in the cell.

In certain embodiments, the present technology is directed to methods of treating a patient, or methods of reducing the appearance of aging in a patient, or methods of optimizing cell maintenance in a patient, comprising applying a composition including sirtuin stimulator and a sirtuin-offsetting agent to the body of a patient.

### BRIEF DESCRIPTION OF THE DRAWINGS

FIG. 1 shows the experimental results of testing regarding the ability of prototype formulation to enhance the apoptotic response of human keratinocytes that had been exposed to DNA-damaging UV radiation.

FIG. 2 shows a mechanism by which the compositions herein may affect sirtuin activity.

### DETAILED DESCRIPTION

Sirtuin function appears to be affected by metabolic state. More broadly, sirtuins appear to help individual cells (and therefore organisms overall) to survive stress, likely by effecting a delay in the apoptotic response, so as to allow cells the time and opportunity to repair whatever stress-related damage they may suffer. It is presumed that any lengthening of lifetime of a cell that results from enhanced sirtuin activity is a consequence of delayed apoptosis under stressful conditions. The cells of greatest interest in this regard would be stem cells. It is believed that the anti-aging effect of sirtuins is at least partly dependent on maintaining the regenerative capacity of such cells and of the tissues they support.

However, as mentioned above, there is concern that the anti-aging mechanism(s) of sirtuins may present an inherent danger. For example, they may impair the normal apoptotic response that would otherwise eliminate damaged cells, and that by doing so, could encourage overgrowth of abnormal cells and tissues. Further, molecular consequences of sirtuin stimulation, such as down regulation of the tumor suppressor gene p53, may also be a cause for concern about enhancing sirtuin function.

In order to offset the negative effects of sirtuins, “sirtuin-offsetting” agents have been proposed. As used herein, a “sirtuin-offsetting” agent (or “sirtuin-offsetter”) is one that at least partly counteracts the effects of sirtuin as it relates to prolonging cell life. Examples of “sirtuin-offsetting” agents will be discussed in greater detail herein. For example, it has been discovered herein that the decreased activity of certain non-sirtuin HDACs may provide a safeguard. In particular, in certain embodiments of the technology described herein, it has been shown that Class 1, 2 and 4 HDACs have a fundamentally different molecular mechanism and different overall biological effects from the sirtuins, which are Class 3 HDACs. These “non-sirtuin” HDACs do not use NAD as a co-substrate. They also differ from the sirtuins in other ways, e.g., in terms of their target proteins and regulation. Anti-aging or life extension effects have not been demonstrated as consequences of stimulation of non-sirtuin HDACs.

It has been found herein that these HDACs, which provide anti-inflammatory benefits, can be targeted to prevent proliferation of genetically damaged or unstable cells. Inhibitors of non-sirtuin HDACs (HDACi's) have been found to be effective at inhibiting proliferation and promoting differentiation or apoptosis and may be useful for cancer treatment, because, among other reasons, in cancer cells many important genes are abnormally repressed by extreme levels of histone deacetylation; thus, the genes that would otherwise be controlling proliferation and initiating differentiation or apoptosis may be inactivated by the non-sirtuin HDACs.

Therefore, in certain embodiments, the present technology is directed to compositions that both stimulate Sirt1 activity (the human sirtuin that is the homolog of yeast Sir2) in order to obtain anti-aging benefits, and also simultaneously inhibit sirtuin-offsetting agents such as, e.g., the non-sirtuin HDACs to the extent sufficient to avoid poorly controlled growth of damaged cells that could eventually compromise tissue function.

In certain embodiments, the compositions herein provide the dual effect of sirtuin stimulation and inhibition of non-sirtuin protein deacetylase (HDAC) activities, which are not necessarily limited to histones as their targets. That is, histones are proteins but the activity in question can be effective with respect to more than just histone functions. Moreover, as used herein, “protein deacetylase” and “protein deacetylation” refer to histone proteins and to all proteins affected by modulation of acetylation state, not necessarily just histones. Examples of proteins that may be useful targets in this regard include the p53, NF-κB and FOXO transcription factors, the PGC1-α transcriptional coactivator, the DNA repair factor ku70, the AMP-activated protein kinase, LKB1, and the cAMP-responsive coactivator TORC2, among others.

Thus, in certain embodiments, the relative amounts of sirtuin activity (or sirtuin stimulator) and sirtuin-offsetting agent in a composition according to the present embodiments is balanced in order to optimize the cell maintenance of the patient. As used herein, “cell maintenance” means the balance of prolonging the life of cells without over-prolonging that can lead to proliferation of damaged tissue and harm to the patient.

In certain embodiments, the present technology provides methods for optimizing the preservation of body cells, such as epidermal keratinocytes, dermal fibroblasts and others, by balancing the Sirt1 activity of a sirtuin composition with the inhibitory activity of an HDACi.

Data have been developed herein that demonstrate, through in vitro studies, the ability to inhibit preferentially the growth of cells that are genetically damaged by exposure to UV light using a formulation that includes activity for inhibiting non-sirtuin HDACs. Cells were subjected to a sub-lethal dose of UV, but by applying a composition in accordance with certain embodiments herein, inhibition of further growth was shown. The effect was to provide time for the cells to repair, and if not, hold them in a quasi-senescent state until they expired.

In various embodiments the compositions herein may comprise any of the following ingredients:

Water, for example, deionized water, in amounts of about 5 to about 50%, about 10 to about 25% or about 15 to about 20%.

An organic solvent, for example, ethanol or a glycol ether (such as phenoxyethanol, ethylene glycol, propylene glycol or diethylene glycol), in amounts of about 10 to about 50%, about 15 to about 40% or about 15 to about 25%; in certain embodiments, two or more organic solvents in a total amount in such ranges;

A nonionic solubilizer or emulsifying agent having an HLB of about 12 to about 16, for example, a hydroxystearate, in amounts of about 10 to about 25% or about 12 to about 20%;

A botanical extract in amounts of about 20 to about 75%, about 25 to about 60% or about 30 to about 55%.

In various embodiments, suitable botanical extracts include extracts from plants (herbs, roots, flowers, fruits, vegetables, seeds, leaves, pollen, nectar); for example, yeast ferment extract, padica pavonica extract, thermus thermophilis ferment extract, camelina sativa seed oil, boswellia serrata extract, olive extract, aribodopsis thaliana extract, acacia dealbat aextract, acer saccharinum (sugar maple), acidophilus, acorus, aesculus, agaricus, agave, agrimonia (for example, Agrimonia species such as Agrimonia eupatoria, Agrimonia gryposepala, Agrimonia incise, Agrimonia coreana, Agrimonia microcarpa, Agrimonia nipponica, Agrimonia parviflora, Agrimonia pilosa, Agrimonia procera, Agrimonia pubescens, Agrimonia repens, Agrimonia rostellata or Agrimonia striata), algae, aloe, citrus, brassica, cinnamon, orange, apple, blueberry, cranberry, peach, pear, lemon, lime, pea, seaweed, caffeine, green tea, chamomile, willowbark, mulberry, poppy, and any other type of botanical extract Further examples include, but are not limited to, Glycyrrhiza Glabra, Salix Nigra, Macrocycstis Pyrifera, Pyrus Malus, Saxifraga Sarmentosa, Vilis Vinifera, Morus Nigra, Scutellaria Baicalensis, Anthemis Nobilis, Salvia Sclarea, Rosmarinus Officianalis, Citrus Medica Limonum, Panax Ginseng, and mixtures thereof.

In certain embodiments, the compositions herein may comprise resveratrol in amounts of about 1 to about 10%, about 2 to about 5% or about 2.5 to about 4%.

In certain embodiments, the compositions herein may comprise either a sirtuin itself, or a sirtuin stimulator, for example, resveratrol or derivatives thereof. In certain embodiments, the compositions are particularly useful for applying to the skin of a patient. In certain embodiments, two active compounds may be combined, for example, a sirtuin stimulator including resveratrol or a composition that behaves like resveratrol; and non-sirtuin histone deacetylase inhibitor (HDACi) as the sirtuin-offsetting agent.

### EXAMPLE 1

An exemplary formulation in accordance with the embodiments hereof was prepared as followed:

Deionized water about 12 to about 20%. Zemea propanediol about 10 to about 20%. Phenoxyethanol about 0.25 to about 1%. Solutol HS 15 about 10 to about 20%. Resveratrol about 1 to about 10%. Botanical extract about 45 to about 55%.

In this Example, the water and organic solvents were combined while stirring and heading to about 40 to about 80 degrees C. the Solutol HS15 and resveratrol were combined thereto with continued stirring. All ingredients were mixed until uniform. The botanical extract was then added to the mixture.

### Example 2

Testing was performed to determine the ability of a prototype formulation in accordance with the present technology, to enhance the apoptotic response of human keratinocytes that had been exposed to DNA-damaging UV radiation. The cells were pretreated with 1% prototype composition for one hour. After washing the cells, they were exposed to 20 mJ/cm² of UVB light. The cells were then treated for another 21 hours with 1% prototype composition. Cell viability was then measured using the Alamar Blue metabolism method, and results were compared to the viability of unirradiated cells treated similarly with the prototype composition.

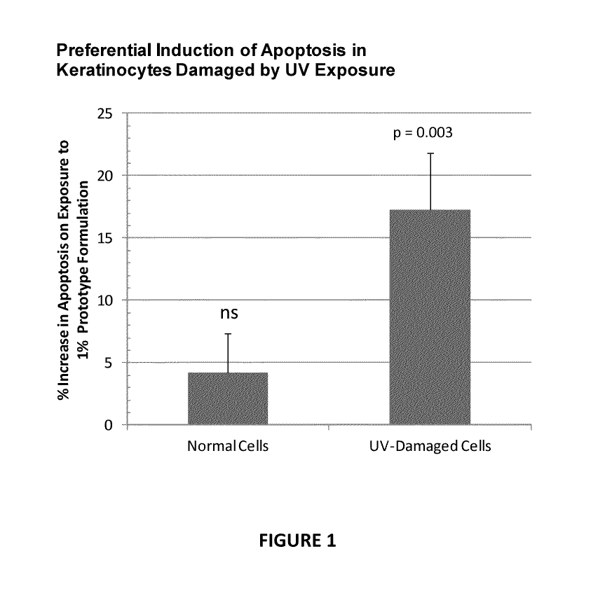

Results are shown in FIG. 1. The graph shows that while the prototype formulation in accordance with the present technology had unmeasurable significant effect on normal unirradiated cells, it did increase apoptosis fourfold in cells damaged by UV exposure. This preferential toxicity toward cells with DNA damage suggests that the composition helps to eliminate repair-deficient (and potentially dangerous) cells, thereby ensuring the survival of a more normal, healthy and proliferating cell population. This effect is conducive to detoxification and to the maintenance of vital, young looking skin.

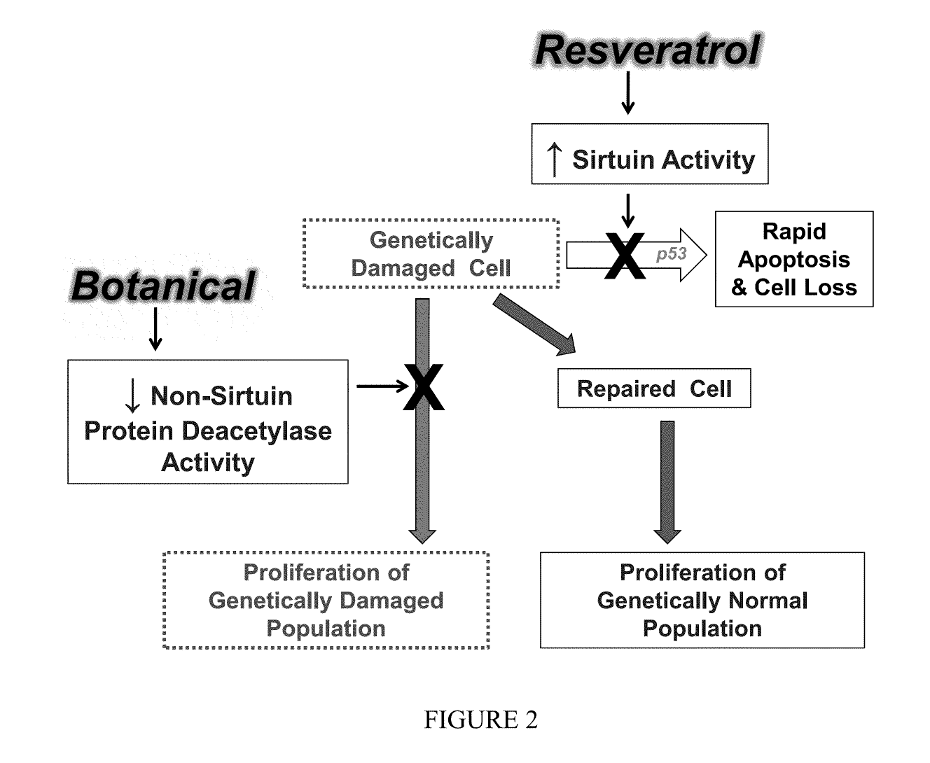

FIG. 2 is a schematic that shows a mechanism by which the compositions herein may affect sirtuin activity. In certain embodiments, the compositions herein comprise both resveratrol and a botanical, and provide a balance between the sirtuin activity and the non-sirtuin protein deacetylase activity. As can be seen, the methods herein provide for an environment in which a genetically damaged cell is less likely to survive and be proliferated, and a repaired or healthy cell is more likely to survive and be proliferated.

The compositions discussed herein may be in any form that can be applied to the body of a patient; for example, to the skin. In certain embodiments, they may be cosmetically or pharmaceutically acceptable forms that can be incorporated into lotions, creams, sprays, gels, ointments, serums, liquids, suspensions or the like. Encapsulation technologies such as liposomes, micellar constructs, polymer and molecular entrapment and the like are also contemplated.

What is claimed:

1. A composition comprising:
(a) a sirtuin stimulator; and
(b) a sirtuin-offsetting agent.

2. The composition of claim 1, wherein the amounts of (a) and (b) are selected to optimize the cell maintenance in a patient.

3. The composition of claim 1, wherein (b) comprises a protein deacetylase inhibitor.

4. A cosmetic composition comprising:
(c) a first composition that comprises a sirtuin stimulator; and
(d) a second composition that comprises a sirtuin-offsetting agent.

5. The composition of claim 4 in the form of a gel, cream, spray, suspension, liquid, paste, ointment or lotion.

6. A method of formulating a composition, the method comprising:
(a) selecting a sirtuin or sirtuin stimulator having a known quantitative prolonging effect on a cell's life; and
(b) selecting a sirtuin-offsetting agent having a known quantitative opposite effect on the cell's life; and
(c) optimizing the balance between the two effects based on a known desired ultimate effect on the cell.

7. A method of prolonging the life of a cell and simultaneously avoiding proliferation of cell damage, the method comprising the steps of:
(a) stimulating Sirt1 activity in the cell; and
(b) inhibiting a non-sirtuin HDAC in the cell.
